(19) United States
(12) Patent Application Publication
(10) Pub. No.: US 2024/0301037 A1
(43) Pub. Date: Sep. 12, 2024

(54) CHIMERIC ANTI-CORONAVIRUS SPIKE PROTEIN ANTIBODIES

(71) Applicant: LA JOLLA INSTITUTE FOR IMMUNOLOGY, La Jolla, CA (US)

(72) Inventors: Erica Ollmann SAPHIRE, La Jolla, CA (US); Kathryn HASTIE, La Jolla, CA (US); Stephanie HARKINS, La Jolla, CA (US)

(21) Appl. No.: 18/272,030

(22) PCT Filed: Jan. 24, 2022
(86) PCT No.: PCT/US2022/013556

§ 371 Date: Jul. 12, 2023

Related U.S. Application Data
(60) Provisional application No. 63/140,516, filed on Jan. 22, 2021.

(51) Int. Cl. C07K 16/10; A61K 39/00; A61P 37/04

(52) U.S. Cl. CPC C07K 16/1003; A61P 37/04; A61K 2039/505; C07K 2317/33; C07K 2317/76; C07K 2317/92

(57) ABSTRACT

Compositions and methods are disclosed for treating and/or preventing coronavirus infection and related pathologies. The compositions include antibodies having certain defined heavy and light chain sequences that recognize the coronavirus spike protein or mutants thereof.

### CROSS-REFERENCE TO RELATED APPLICATION

This application claims priority to, and the benefit of, U.S. Provisional Patent Application Ser. No. 63/140,516 filed Jan. 22, 2021, entitled “CHIMERIC ANTI-SARS-COV2 SPIKE PROTEIN ANTIBODIES”, the entire disclosure of which is incorporated herein by reference in its entirety.

### TECHNICAL FIELD

The present application relates to antibodies, and particularly to antibodies that recognize the spike protein of coronaviruses, including SARS-COV-2.

### BACKGROUND

Effective countermeasures against the emergence and expansion of the 2019-Novel Coronavirus (SARS-COV-2) require the development of new tools for monitoring and treatment.

As of January 2022, more than 350 million people have been infected with or succumbed to diseases associated with SARS-COV-2. As such, the aforementioned tools are urgently required.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, an aspect of the present disclosure relates to an antibody composition that recognizes the spike protein or mutants thereof of a coronavirus.

As embodied and broadly described herein, an aspect of the present disclosure relates to an antibody composition that recognizes the SARS-COV-2 spike protein or mutants thereof.

In an aspect, the antibody composition comprises a heavy chain having at least 80%, or at least 85%, or at least 90%, or at least 95% sequence identity with any one of SEQ ID NOs: 1-57. In embodiments, the heavy chain comprises any one of SEQ ID NOs: 1-57.

In another aspect, an antibody composition is disclosed comprising a light chain having at least 80%, or at least 85%, or at least 90%, or at least 95% sequence identity with any one of SEQ ID NOs: 58-114. In embodiments, the light chain comprises any one of SEQ ID NOs: 58-114.

In another aspect, an antibody composition is disclosed comprising a heavy chain having at least 80%, or at least 85%, or at least 90%, or at least 95% sequence identity with any one of SEQ ID NOs: 1-57; and a light chain having at least 80%, or at least 85%, or at least 90%, or at least 95% sequence identity with any one of SEQ ID NOs: 58-114. In embodiments, the heavy chain comprises any one of SEQ ID NOs: 1-57; and the light chain comprises any one of SEQ ID NOs: 58-114.

In embodiments, any of the antibody compositions described herein are specific for a spike protein of a coronavirus. In another aspect, the coronavirus is SARS, MERS, 229E (alpha), NL63 (alpha), OC43 (beta), HKU1 (beta), SARS-COV-2, or an emerging variant or sub-strain thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or the Omicron sub-strain BA.2. In embodiments, any of the antibody compositions described herein are specific for a spike protein of SARS-COV-2. In embodiments, any of the antibody compositions described herein are specific for a receptor binding domain of SARS-COV-2. In embodiments, any of the antibody compositions described herein are specific for an N-terminal domain of SARS-COV-2.

In another aspect, a method of treating an individual infected with a coronavirus is disclosed. The method involves administering to the individual an effective amount of an antibody composition as detailed herein.

In another aspect, a method of treating an individual infected with SARS-COV-2 is disclosed. The method involves administering to the individual an effective amount of an antibody composition as detailed herein.

In another aspect, a method of preventing a coronavirus infection in an individual is disclosed. The method involves administering to the individual an effective amount of an antibody composition as detailed herein.

In another aspect, a method of preventing SARS-COV-2 infection in an individual is disclosed. The method involves administering to the individual an effective amount of an antibody composition as detailed herein.

In embodiments, in any of the methods of treating or preventing coronavirus described herein, the antibody composition binds to a receptor binding domain of the coronavirus. In embodiments, in any of the methods of treating or preventing coronavirus infection described herein, the antibody composition binds to a spike protein of the coronavirus. In embodiments, in any of the of the methods of treating or preventing coronavirus infection described herein, the antibody composition neutralizes coronavirus pseudovirions.

In embodiments, in any of the methods of treating or preventing SARS-COV-2 described herein, the antibody composition binds to a receptor binding domain of SARS-COV-2. In embodiments, in any of the methods of treating or preventing SARS-COV-2 infection described herein, the antibody composition binds to a spike protein of SARS-COV-2. In embodiments, in any of the of the methods of treating or preventing SARS-COV-2 infection described herein, the antibody composition neutralizes SARS-COV-2 pseudovirions.

In another aspect, a method of identifying SARS-COV-2 in an individual is disclosed. The method involves isolating a biological sample from the individual; incubating the biological sample with an antibody composition as detailed herein; and detecting a biological interaction between the biological sample and the antibody composition.

In another aspect, a kit for identifying SARS-COV-2 in a biological sample is disclosed. The kit includes an antibody composition as detailed herein; and instructions of use.

In another aspect, an enzyme-linked immunosorbent assay (ELISA) test kit is disclosed. The ELISA test kit includes an antibody composition as detailed herein; and instructions of use.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE FIGURES

In the present application:

FIG. 1 demonstrates certain ELISA binding data as detailed herein.

FIG. 2 demonstrates neutralization of SARS-CoV2 pseudovirions as detailed herein.

FIGS. 3A-3B demonstrate binding and kinetic analysis of anti-coronavirus spike antibodies as detailed herein.

FIGS. 4A-4B demonstrate neutralization of SARS-CoV-2 pseudovirions by select antibodies as detailed herein.

FIGS. 5A-5C demonstrate structural characterization of anti-coronavirus spike antibodies as detailed herein.

FIG. 6 demonstrates protection of K18-hACE2 tg mice against SARS-COV-2 infection as detailed herein.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Overview of Disclosure

The present disclosure describes antibodies that recognize coronavirus spike protein. The disclosure also describes certain methods of treatment, both for therapeutic and prophylactic purposes.

### Definitions and Interpretation

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

If and as used herein, the term “administering”, when used in relation to an expression vector, nucleic acid molecule, or a delivery vehicle (such as a chitosan nanoparticle) to a cell, refers to transducing, transfecting, electroporation, translocating, fusing, phagocytosing, shooting or ballistic methods, etc., i.e., any means by which a protein or nucleic acid can be transported across a cell membrane and preferably into the nucleus of a cell.

Unless otherwise indicated, a particular amino acid sequence that is recited herein also implicitly encompasses conservatively modified variants. As a non-limiting example, any of the disclosed amino acid sequences for portions of an anti-SARS-COV-2 spike protein antibody implicitly encompass conservatively modified variants.

If and as used herein, the terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. The expression “amino acid analogs” refers to compounds that have the same basic chemical structure as a naturally occurring amino acid, i.e., an alpha. carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

If and as used herein, the term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions or additions that alter, add or delete a single amino acid or -nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art.

For example, the following groups each contain amino acids that are conservative substitutions for one another (see, e.g., Creighton, Proteins (1984) W.H. Freeman, New York, pages 6-20, for a discussion of amino acid properties):

Alanine (A), Glycine (G)
Serine (S), Threonine (T)
Aspartic acid (D), Glutamic acid (E)
Asparagine (N), Glutamine (Q)
Cysteine (C), Methionine (M)
Arginine (E), Lysine (K), Histidine (H)
Isoleucine (I), Leucine (L), Valine (V)
Phenylalanine (F), Tyrosine (Y), Tryptophan (W)

In light of the present disclosure, in particular in view of the experimental data described in the examples of the present text, the person of skill will readily understand which amino acid may be substituted, deleted or added to a given sequence to create a conservatively modified variant comprising an amino acid sequence which is at least at least 95%, or at least 96%, or at least 97%, or at least 98%, or at least 99%, identical to one or more amino acid sequence set forth in the table above without undue effort.

If and used herein, the terms “treating” or “treatment” refers to a process by which an infection, such as infection with a coronavirus, such as SARS-COV-2, or a disease or the symptoms of an infection or a disease associated with a viral strain are prevented, alleviated or completely eliminated. As used herein, the term “prevented” or “preventing” refers to a process by which an infection or a disease or symptoms of an infection or a disease associated with a virus, such as SARS-CoV-2, are averted prior to infection.

If and as used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

If and used herein, the expression “an acceptable carrier” refers to a vehicle for containing a compound that can be administered to a subject without significant adverse effects.

As used herein, the term “adjuvant” means a substance added to the composition of the invention to increase the composition's immunogenicity. The mechanism of how an adjuvant operates is not entirely known. Some adjuvants are believed to enhance the immune response (humoral and/or cellular response) by slowly releasing the antigen, while other adjuvants are strongly immunogenic in their own right and are believed to function synergistically.

With respect to the present disclosure, an adjuvant may be selected from aluminum hydroxide or mineral oil, and a stimulator of immune responses, such as Bordatella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In an embodiment, adjuvants may include adjuvants which stimulate a Thl type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvants.

If and used herein, the terms “determining,” “measuring,” “evaluating,” “assessing,” and “assaying” generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

If and used herein, the expression “biological sample” includes, in the present disclosure, any biological sample that is suspected of comprising a T cell, such as for example but without being limited thereto, blood and fractions thereof, urine, excreta, semen, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like. In one non-limiting embodiment, a herein described biological sample can be obtained by any known technique, for example by drawing, by non-invasive techniques, or from sample collections or banks, etc.

If and used herein, the expression “treatment” includes inducing, enhancing, or sustaining an immune response against a viral infection or symptoms associated thereto. For example, the treatment may induce, increase, promote or stimulate anti-viral virus activity of immune system cells in a subject following the treatment. In non-limiting examples, the immune system cells may include adaptive immune cells, such as T cells, including CD4⁺ T cells, CD8⁺ T cells, and/or B cells, or innate immune cells, such as macrophages and/or neutrophils.

If and used herein, the expression “therapeutically effective amount” may include the amount necessary to allow the component or composition to which it refers to perform its immunological role without causing overly negative effects in the host to which the component or composition is administered. The exact amount of the components to be used or the composition to be administered will vary according to factors such as the type of condition being treated, the type and age of the subject to be treated, the mode of administration, as well as the other ingredients in the composition.

As used herein, the term “virus” generally refers to a coronavirus. In particular embodiments, the virus is a betacoronavirus. In another embodiment, the coronavirus is SARS, MERS, 229E (alpha), NL63 (alpha), OC43 (beta), HKU1 (beta), SARS-COV-2, or an emerging variant or sub-strain thereof. SARS-COV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or the Omicron sub-strain BA.2. In still other embodiments, the virus is SARS-COV-2 or any mutant form thereof. As used herein, the term “SARS-COV-2” refers to all phylogenetic samples of SARS-COV-2 genomes, as well as any and all mutant or variant strains thereof. As used herein, the term “spike protein”, which may also be referred to as “S protein”, refers to a protein expressed by SARS-COV-2, and includes, without limitation, reference to SARS-COV-2 spike protein (Severe acute respiratory syndrome coronavirus 2 spike protein, 2019 novel coronavirus spike protein, SARS-COV-2 spike protein, SARS-COV-2 spike protein, COVID-19).

Other examples of implementations will become apparent to the person skilled in the art in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

### Detailed Description of Aspects and Embodiments of the Disclosure

As embodied and broadly described herein, an aspect of the present disclosure relates to an antibody composition comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 1-57. In embodiments, the heavy chain comprises any one of SEQ ID NOs: 1-57.

In another aspect, an antibody composition is disclosed comprising a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 58-114. In embodiments, the light chain comprises any one of SEQ ID NOs: 58-114.

In another aspect, an antibody composition is disclosed comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 1-57; and a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 58-114. In embodiments, the heavy chain comprises any one of SEQ ID NOs: 1-57; and the light chain comprises any one of SEQ ID NOs: 58-114.

In In embodiments, any of the antibody compositions described herein are specific for a spike protein of SARS-COV-2. In embodiments, any of the antibody compositions described herein are specific for a receptor binding domain of SARS-COV-2. In embodiments, any of the antibody compositions described herein are specific for an N-terminal domain of SARS-COV-2

The above-mentioned antibody compositions also include any and all variants, modifications, homologues, derivatives or subsequences thereof.

In another aspect, a method of treating an individual infected with SARS-COV-2 is disclosed. The method involves administering to the individual an effective amount of an antibody composition as detailed herein.

In another aspect, a method of preventing SARS-COV-2 infection in an individual is disclosed. The method involves administering to the individual an effective amount of an antibody composition as detailed herein.

In embodiments, in any of the methods of treating or preventing SARS-COV-2 described herein, the antibody composition binds to a receptor binding domain of SARS-COV-2. In embodiments, in any of the methods of treating or preventing SARS-COV-2 infection described herein, the antibody composition binds to a spike protein of SARS-COV-2. In embodiments, in any of the of the methods of treating or preventing SARS-COV-2 infection described herein, the antibody composition neutralizes SARS-COV-2 pseudovirions.

In another aspect, a method of identifying SARS-COV-2 in an individual is disclosed. The method involves isolating a biological sample from the individual; incubating the biological sample with an antibody composition as detailed herein; and detecting a biological interaction between the biological sample and the antibody composition.

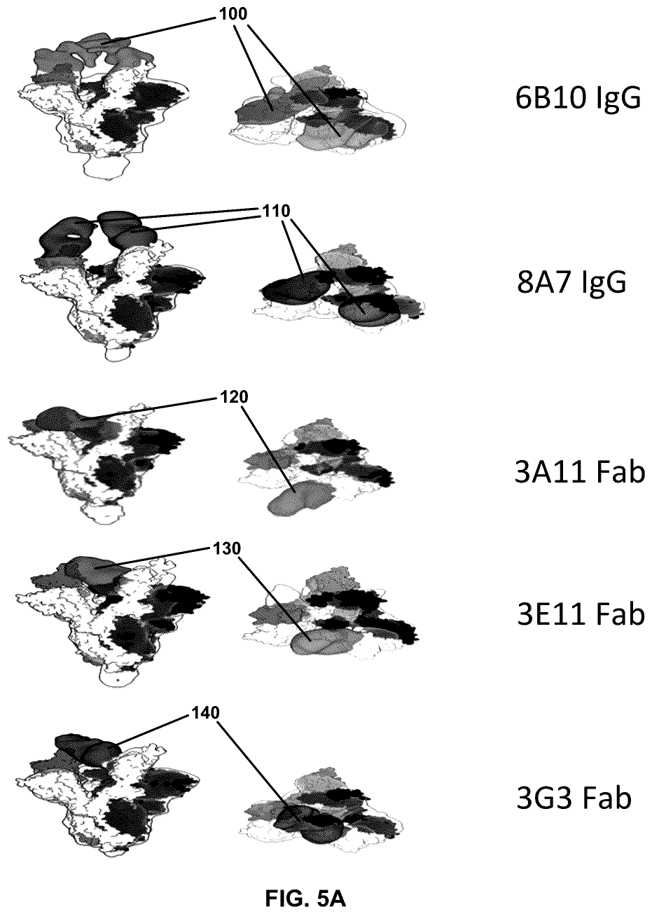

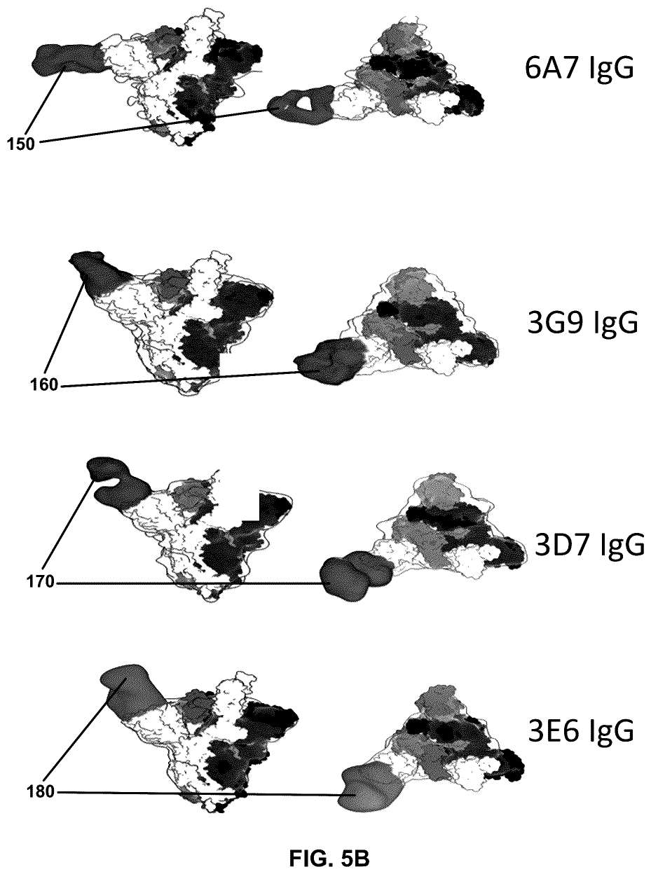

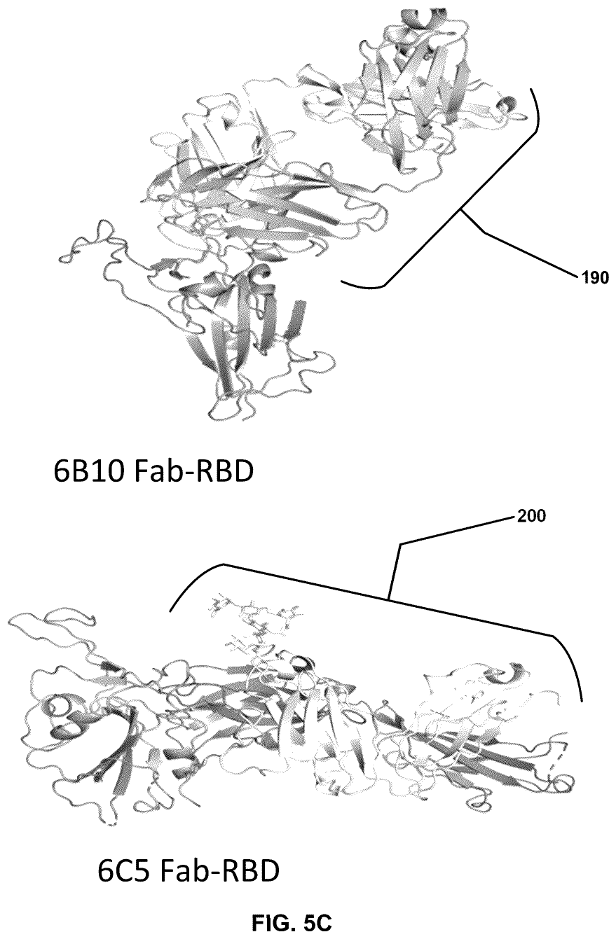

FIGS. 5A-5C illustrate embodiments of structural characterizations of various anti-SARs-CoV-2 bound to various SARs spike proteins. FIG. 5A illustrates embodiments of binding of antibodies and fragment antigen binding (Fab) regions of antibodies to a trimeric spike protein of SARs-COV-2. Element 100 illustrates an embodiment of the 6B10 IgG antibody as it is bound to the trimeric spike protein of SARS-COV-2. Element 110 illustrates an embodiment of the 8A7 IgG antibody as it is bound to the trimeric spike protein of SARS-COV-2. Element 120 illustrates an embodiment of the 3A11 Fab as it is bound to the trimeric spike protein of SARS-COV-2. Element 130 illustrates an embodiment of the 3E11 Fab as it is bound to the trimeric spike protein of SARS-CoV-2. Element 140 illustrates an embodiment of the 3G3 Fab as it is bound to the trimeric spike protein of SARS-COV-2.

FIG. 5B illustrates embodiments of N-terminal domain (NTD) specific antibodies bound to a trimeric spike protein of SARS-COV-2. Element 150 illustrates an embodiment of the 6A7 IgG antibody as it is bound to the trimeric spike protein of SARS-COV-2. Element 160 illustrates an embodiment of the 3G9 IgG antibody as it is bound to the trimeric spike protein of SARS-COV-2. Element 170 illustrates an embodiment of the 3D7 IgG antibody as it is bound to the trimeric spike protein of SARS-COV-2. Element 180 illustrates an embodiment of the 3E6 IgG antibody as it is bound to the trimeric spike protein of SARS-COV-2.

FIG. 5C illustrates embodiments of crystal structures of Fab regions bound to a receptor binding domain (RBD) of SARS-COV-2. Element 190 illustrates an embodiment of the 6B10 Fab as it is bound to the RBD of SARS-COV-2. Element 200 illustrates an embodiment of the 6C5 Fab as it is bound the RBD of SARS-COV-2.

In another aspect, a kit for identifying SARS-COV-2 in a biological sample is disclosed. The kit includes an antibody composition as detailed herein; and instructions of use.

In another aspect, an enzyme-linked immunosorbent assay (ELISA) test kit is disclosed. The ELISA test kit includes an antibody composition as detailed herein; and instructions of use.

In accordance with the invention, treatment methods are provided that include therapeutic (following infection with SARS-COV-2) and prophylactic (prior to SARS-COV-2 exposure, infection or pathology) methods. For example, therapeutic and prophylactic methods of treating a subject for a viral infection include treatment of a subject having or at risk of having a viral infection or pathology, treating a subject with a viral infection, and methods of protecting a subject from a viral infection, to decrease or reduce the probability of a viral infection in a subject, to decrease or reduce susceptibility of a subject to a viral infection, or to inhibit or prevent a viral infection in a subject, and to decrease, reduce, inhibit or suppress transmission of a virus from a host to a subject.

Such methods include the administration of the antibodies disclosed herein to therapeutically or prophylactically treat a subject having or at risk of having a virus infection or pathology. Accordingly, methods can treat the virus infection or pathology, or provide the subject with protection from infection (e.g., prophylactic protection).

In particular embodiments, one or more disorders, diseases, physiological conditions, pathologies and symptoms associated with or caused by SARS-COV-2 viral infection or pathology will respond to treatment. In particular methods embodiments, treatment methods reduce, decrease, suppress, limit, control or inhibit viral numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a viral nucleic acid. In additional particular methods embodiments, treatment methods include an amount of an antibody composition, subsequence or portion thereof sufficient to increase, induce, enhance, augment, promote or stimulate an immune response against a virus; increase, induce, enhance, augment, promote or stimulate viral clearance or removal; or decrease, reduce, inhibit, suppress, prevent, control, or limit transmission of a particular virus to a subject (e.g., transmission from a host to a subject). In further particular methods embodiments, treatment methods include an amount of an antibody composition, subsequence or portion thereof sufficient to protect a subject from a viral infection or pathology, or reduce, decrease, limit, control or inhibit susceptibility to viral infection or pathology.

Methods of the present disclosure include treatment methods, which result in any therapeutic or beneficial effect. In various methods embodiments, viral infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with a viral infection, proliferation or replication, or pathology. In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with a particular viral infection, proliferation or replication, or pathology. In further various particular embodiments, treatment methods include improving, accelerating, facilitating, enhancing, augmenting, or hastening recovery of a subject from a particular viral infection or pathogenesis, or one or more adverse symptoms, disorders, illnesses, diseases or complications caused by or associated with a viral infection, proliferation or replication, or pathology. In yet additional various embodiments, treatment methods include stabilizing infection, proliferation, replication, pathogenesis, or an adverse symptom, disorder, illness, disease or complication caused by or associated with a viral infection, proliferation or replication, or pathology, or decreasing, reducing, inhibiting, suppressing, limiting or controlling transmission of a virus from and to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with a viral infection, proliferation or replication, or pathology. Thus, a satisfactory clinical endpoint is achieved when there is an incremental improvement or a partial reduction in an adverse symptom, disorder, illness, disease or complication caused by or associated with a viral infection, proliferation or replication, or pathology, or an inhibition, decrease, reduction, suppression, prevention, limit or control of worsening or progression of one or more adverse symptoms, disorders, illnesses, diseases or complications caused by or associated with a particular viral infection, viral numbers, titers, proliferation or replication, viral protein or nucleic acid, or viral pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a viral infection or pathology. For example, reducing an amount of an adjunct therapy, for example, a reduction or decrease of a treatment for a viral infection or pathology, or a vaccination or immunization protocol is considered a beneficial effect. In addition, reducing or decreasing an amount of a viral antigen used for vaccination or immunization of a subject to provide protection to the subject is considered a beneficial effect.

Adverse symptoms and complications associated with a viral infection and pathology include, but are not limited to for example, e.g., fever, rash, headache, cough, shortness of breath or difficulty breathing, chills (including shaking associated with chills), sore throat, loss of taste or smell, tiredness, difficulty breathing, pain behind the eyes, conjunctivitis, muscle or joint pain, nausea, vomiting, loss of appetite, or secondary infection. Other symptoms of a viral infection or pathogenesis are known to one of skill in the art and treatment thereof in accordance with the invention is provided. Thus, the aforementioned symptoms and complications are treatable in accordance with the present disclosure.

Adverse symptoms and complications associated with viral infections and pathologies can include, but are not limited to, for example, e.g., fever, rash, headache, cough, tiredness, difficulty breathing, pain including pain behind the eyes, stomach pain, muscle or joint pain, weakness, fatigue, conjunctivitis, nausea, vomiting, diarrhea, loss of appetite, hemorrhaging, bleeding, bruising, or secondary infection. Other symptoms of viral infections or pathogenesis are known to one of skill in the art and treatment thereof in accordance with the invention is provided. Thus, the aforementioned symptoms and complications are treatable in accordance with the present disclosure.

Methods, uses and compositions of the present disclosure include administration of the antibody compositions to a subject prior to contact, exposure or infection by a particular virus, administration prior to, substantially contemporaneously with or after a subject has been contacted by, exposed to or infected with a particular virus, and administration prior to, substantially contemporaneously with or after a particular virus pathology or development of one or more adverse symptoms, disorders, illness or diseases caused by or associated with a particular viral infection, or pathology. A subject infected with a particular virus may have an infection over a period of 1-5, 5-10, 10-20, 20-30, 30-50, 50-100 hours, days, months, or years.

Compositions and uses and methods of the present disclosure can be combined with any compound, agent, drug, treatment or other therapeutic regimen or protocol having a desired therapeutic, beneficial, additive, synergistic or complementary activity or effect.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization.

Methods of the present disclosure also include, among other things, methods that result in a reduced need or use of another compound, agent, drug, therapeutic regimen, treatment protocol, process, or remedy. For example, for a particular viral infection or pathology, vaccination or immunization, a method of the present disclosure has a therapeutic benefit if in a given subject a less frequent or reduced dose or elimination of an anti-viral treatment results. Thus, in accordance with the invention, methods of reducing need or use of a treatment or therapy for a particular viral infection or pathology, or vaccination or immunization, are provided.

Without limiting any of the foregoing, the following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The compositions of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For instance, the composition of the present disclosure may be administered in the form of an injectable preparation, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. Suitable dosages will vary, depending upon factors such as the amount of each of the components in the composition, the desired effect (short or long term), the route of administration, the age and the weight of the subject to be treated. Any other methods well known in the art may be used for administering the composition of the present disclosure.

The antibody compositions of the present disclosure may be formulated as a dry powder (i.e . . . in lyophilized form). Freeze-drying (also referred to as lyophilisation) is often used for preservation and storage of biologically active material because of the low temperature exposure during drying. Lyophilizing the composition can result in a more stable composition.

In certain embodiments, the composition of the present disclosure may be formulated as a liquid (e.g., aqueous formulation), e.g., as syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g . . . lecithin or acacia); non-aqueous vehicles (e.g., almond oil, oily esters, or fractionated vegetable oils); and preservatives (e.g., methyl or propyl-p-hydroxybenzoates or sorbic acid).

Where the composition of the present disclosure is intended for delivery to the respiratory (e.g., nasal) mucosa, typically it is formulated as an aqueous solution for administration as an aerosol or nasal drops, or alternatively, as a dry powder, e.g., for rapid deposition within the nasal passage. Compositions for administration as nasal drops may contain one or more excipients of the type usually included in such compositions, for example preservatives, viscosity adjusting agents, tonicity adjusting agents, buffering agents, and the like. Viscosity agents can be microcrystalline cellulose, chitosan, starches, polysaccharides, and the like. Compositions for administration as dry powder may also contain one or more excipients usually included in such compositions, for example, mucoadhesive agents, bulking agents, and agents to deliver appropriate powder flow and size characteristics. Bulking and powder flow and size agents may include mannitol, sucrose, trehalose, and xylitol.

### EXAMPLES

### Example 1. ELISA Binding Data

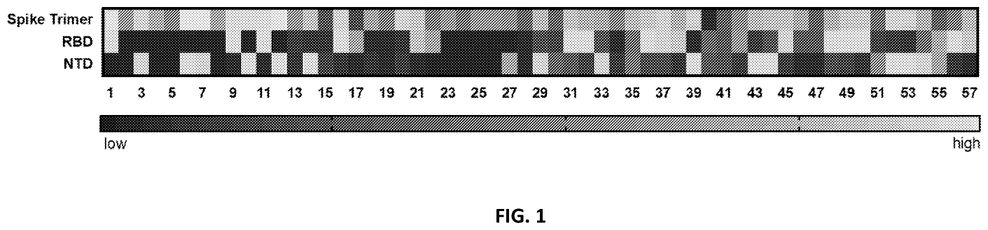

SARS-COV-2 spike ectodomain, receptor binding domain (RBD) or N-terminal domain (NTD) was coated on ELISA microtiters plates. Supernatants from small-scale (2 mL) antibody test expressions were captured overnight and binding analyzed with an anti-human IgG-HRP secondary. 57 unique antibodies demonstrated binding to one or more of the coated proteins, with subunit-specificity distributed evenly (Spike trimer only, n=20; RBD, n=20; NTD, n=17). The data is summarized in FIG. 1.

Heavy chain sequence corresponding to the tested sequences are described below:

Heavy Chains
001-A7k
(SEQ ID NO: 1)
EVQLQESGPGLVKPSETLALTCTVSGGSTSSYYWSWIRQPPGKGL
EWIGSIYYSGSTDYNPSLKSRVTISLDTSKTQFSLKLSSVTAADT
AVYYCARVDTSGWYTGDVFDYWGQGTLVTVSS
001-A8L
(SEQ ID NO: 2)
EVQLVESGGGLVQPGGSLRLSCAASGFTFSIYSMNWVRQAPGKGL
EWVSYISSGSSTIYYADSLKGRFTISRDNAKNSLYLQMISLRDED
TAVYYCARDGVGPTHYFDYWGQGTLVTVSS
001-A10k
(SEQ ID NO: 3)
QVQLVQSGAEVKKPGSSVKVSCKASGGTFRTYAISWVRQAPGQGL
EWMGGIIPIFGTANYAQKFQGRVTITADKSTSTAYMELTSLRSED
TAVYYCARLSATVLTPSYWGQGTLVTVSS
001-A11L
(SEQ ID NO: 4)
EVQLVESGGGVVQPGRSLRLSCAASGFSFSSYAIYWVRQAPGKGL
EWVALISYDGSNKYYADSVKGRFTISRDNSKNTLYLQMNSLKTED
TAVYYCARPRGGNYIDGFDMWGQGTMVTVSS
001-A12L
(SEQ ID NO: 5)
EVQLVESGGGLVQPGGSRRLSCAASGFSFSSYWMSWVRQAPGKGL
EWVANIKQDGREKYYVDSVKGRFTISRDNAKNSLYLQMNSLRAED
TAVYFCARLGSSSWYFGFWGQGTLVTVSS
996-A7k
(SEQ ID NO: 6)
QVQLVQSGAEVKKPGASVKVSCKASGYTFTGQYMHWVRQAPGQGL
EWMGWINPNSGGTNYAQKFQGRVTMTRDTSISTAYMELSRLRSDD
TAVYYCARWHSYYNFYGMDVWGQGTTVTVSS
996-A11k
(SEQ ID NO: 7)
QVQLVESGGGVVQPGRSLRLSCAASGFTFSNYAMHWVRQAPGKGL
EWVAVTSYDGSNKYYADSVRGRFTISRDNYKNTLYLQMNSLRAED
TAVYYCARDRSAFHGTTVTTEEGFFDYWGQGTLVTVSS
996-Blk
(SEQ ID NO: 8)
QVQLQESSPGLVKPSQTLSLTCTVSGGSISSGSHYWSWIRQPAGK
GLDWIGRIHTSGSTNYNPSLKSRVTISVDTSKNQFSLKLSSVTAA
DTAVYYCARERLVSRYSSGWYNYYYYGMDVWGQGTTVTVSS
996-C5L
(SEQ ID NO: 9)
QVQLVESGGGLVQPGGSLRLSCAASGFTFSYYWMSWVRQAPGKGL
EWVANIKQDGSEKYYVDSVKGRFTISRDNAKNSLYLQMNSLRAED
TAVYYCARDRYGWPSIPAPFDYWGQGTLVTVSS
996-C6k
(SEQ ID NO: 10)
QVQLVQSGAEVKKPGASVKVSCKASGYTFTIYGIHWVRQAPGQRL
EWMGWINAGNGDTKYSQRFQGRVTITRDTSASTAYMELSSLRSED
TAVYYCARGGTMYGLSNYYSYAMDVWGQGTTVTVSS
996-C7k
(SEQ ID NO: 11)
EVQLVESGGGLVQPGGSLRLSCAASGFTFSTYDMHWVRQATGKGL
EWVSAIGTAGDTYYPGSVKGRFTISRENAKNSLYLQMNSLRAGDT
AVYYCARGGSTVTTSFDYWGQGTLVTVSS
996-C9L
(SEQ ID NO: 12)
QVQLVQSGAEVKKPGASVKVSCKVSGYTLTELSMHWVRQAPGKGL
EWMGGFDPEDGETIYAQKFQGRVTMTEDTSTDTAYMELSSLRSED
TAVYYCAAGGPTSYYYDSSGYFRNLDYWGQGTLVTVSS
206-A1L
(SEQ ID NO: 13)
QVQLQQSGGGLIQPGGSLRLSCAASGFIVSSNYMSWVRQAPGKGL
EWVSVIYSGGSTFYADSVKGRFTISRDNSNNTLYLQMNSLRAEDT
AVYYCARDYGDFYFDYWGQGTLVTVSS
206-A8L
(SEQ ID NO: 14)
QMQLVQSGGGVVQPGRSLRLSCAASGFTFSNYGMHWVRQAPGKGL
EWVAVIWYDGSNKYYTDSVKGRFTISRDNSKNTLFLQMNSLRADD
TALYYCARDIPALGDPTEDFHIWGQGTMVTVSS
206-A9k
(SEQ ID NO: 15)
QVQLVESGGGVVRPGRSLRLSCAASGFTFSSYAMHWVRQAPGKGL
EWVAVISYDGSNKYHADSVKGRVTISRDNSKNTLYLQMNSQRADD
TAAYYCARGTGGGYYYGMDVWGQGTTVTVSS
206-B10L
(SEQ ID NO: 16)
QVQLVQPGGGVIQPGRSLRLSCTASGFTFSSYPMHWVRQAPGKGL
EWVAVISYDGDNKYYADSVKGRFTISRDNSKNTLYLQMNSLKSDD
TAMYYCARDYVVVILSDSFYYGMDVWGQGTTVTVSS
206-C8K
(SEQ ID NO: 17)
EVQLLESGGGLVKPGGSLRLSCAASGFTFSSYSMNWVRQAPGKGL
EWVSSISSSSSYIYYADSVKGRFTISRDNAKNSLYLQMNSLRAED
TAVYYCARDTPTEYYDFWSGYYWRAFDIWGQGTMVTVSS
206-C10k
(SEQ ID NO: 18)
EVQLVQSGGGLVQPGGSLRLSCAASGFPLRSYAMSWVRQAPGKGL
EWVSAISGSGGSTYYADSVKGRFSISRDNSKNTLYLQMNSLRAED
TAVYYCAKGLEGVILTGYYIHGMDVWGQGTTVTVSS
206-D5L
(SEQ ID NO: 19)
QVQLVQSGAEVRKPGASVKVSCKASGYTFTGYYIHWVRQAPGQGL
EWMGWINPNSGGTNYAQKFQGWVIMTRDTSISTAYMELSRLTSDD
TAVYYCARCGARSYYYDSGDYCAFDIWGQGTVVTVSS
206-Elk
(SEQ ID NO: 20)
QVQLVQSGAEVKKPGASVKVSCKASGYTFSDYYIHWVRQAPGQGL
EWVGIFNPSGGLTSYAQKFQGRVTMTMDTSTTTVYMELSSLRSED
TAVYFCARDPRFNRNDVADAFDIWGQGTMVTVSS
208-A7k
(SEQ ID NO: 21)
EVQLLESGGGLIQPGGSLRLSCAASGFIVSSNYMSWVRQAPGKGL
EWVSVIYSGGSTFYADSVKGRFTISRDNSNNTLYLQMNSLRAEDT
AVYYCARDYGDFYFDYWGQGTLVTVSS
208-A11k
(SEQ ID NO: 22)
EVQLVQSGAEVKKPGSSVKVSCKASGGTFSSYTISWVRQAPGQGL
EWMGRIIPILDIANYAQRFQGRVTITADKSTSTAYMELSSLRSED
TAVYYCARDLCSSTSCYSHTGFDYWGQGTLVTVSS
208-B2L
(SEQ ID NO: 23)
EVQLVQSGGGVVQPGKSLRLSCATSGFTFSGYGMYWVRQAPGKGL
EWVAVISYDGYNKYYADSVKGRFTISRDNSKNTLYVQMNSLRAED
TAVYYCAKTYSGSYLGYFDYWGQGTLVTVSS
208-C12L
(SEQ ID NO: 24)
QVQLVQSGGVVVQPGGSLRLSCAASGFTFDDYAMFWVRQGPGKGL
EWVSLISWDGTSTYYADSVKGRFTISRDNSKNSLYLQMNSLRTED
TALYYCAKGGRAAIYGMDVWGQGTMVTVFS
208-D3k
(SEQ ID NO: 25)
QVQLVESGGGVVQPGRSLRLSCAASGFTFGTYGIHWVRQAPGKGL
EWVALISYDGTNKYYADSVKGRFTVSRGNSKNTLYLQMNSPRRED
TAVYYCAIPESGSYYQPFDCWGQGTLVTVSS
208-D11L
(SEQ ID NO: 26)
QVQLVQSGGGVVQPGKSLRLSCATSGFTFSGYGMYWVRQAPGKGL
EWVAVISYDGYNKYYADSVKGRFTISRDNSKNTLYVQMNSLRAED
TAVYYCAKTYSGSYLGYFDYWGQGTLVTVSS
208-F1k
(SEQ ID NO: 27)
QVQLLESGPGLVKPSGTLSLTCAVSGGSISSGIWLTWVRQPPGKG
LEWIGEIYHSGSTNYNPSLKSRVSISVDKSKNQFSLNLSSVTAAD
TAVYYCARRKLPLTIDYWGQGTLVTVSS
213-A1L
(SEQ ID NO: 28)
QMQLVQSGGGVVQPGRSLRLSCAASGFTFSTYAMHWVRQAPGKGL
EWVAVISYDGSNKYYADSMKGRFTISRDNSKNTLYLQMNSLRAED
TAVYYCARGNYGSYYYGMDVWGQGTTVTVSS
213-A3L
(SEQ ID NO: 29)
QVQLVQSGGGLVQPGGSLRLSCAASGFTLSSCGMNWVRQAPGKGL
EWVSYISSSSISIYYADSVKGRFTISRDNAKNSLYLQMNSLRAED
TAVYFCARDPPSVAGTWYSYYYMDVWGRGTTVTVSS
213-A5L
(SEQ ID NO: 30)
QVQLLQPGAEVKKPGASVKVSCKASGYTFNSDDINWVRQATGQGL
EWMGWMNPDSGNTGYAQKFQGRVTMTRNTSISTAYMELSSLRSED
TAVYYCAGTVSGYDFHSSYYYIDVWGKGTTVTVSS
213-A9L
(SEQ ID NO: 31)
QMQLVQSGPGLVKPSETLSLTCTVSGGSISSYYWSWIRQPPGKGL
EWIAYMYYSGSTNFNPSLKSRVTISVDTSKNQFSLKLSSVTAADT
AVYYCARARPNLWFGAFDYWGQGTLVTVSS
213-A11L
(SEQ ID NO: 32)
EVQLVQSGGGLVKPGGSLRLSCAASRFTFSDYYMSWIRQAPGKGL
EWVSYISSSGSTIDYADSVKGRFTISRDNAKNSLYLQMNSLRAED
TAVYYCAGSSWLRGWFDPWGQGTLVTVSS
213-B6k
(SEQ ID NO: 33)
QVQLVQSGPGLVKPSETLSLTCTVSGGSISTGNYYWGWIRQPPGK
GLEWIGSIYYSGTTYYNPSLKSRVTISVDTSKNQFSLKLISMTAA
DTAVYYCARHGAPIGISWFGREDWFDPWGQGTLVTVSS
213-B12L
(SEQ ID NO: 34)
QVQLVESGGGVVQPGRSLRLSCAATGFTFSTYAMHWVRQAPGKGL
EWVAFISYDGTNKYYADSVKGRFTISRDNSKNTLYLQMNSLRAED
TAVYYCARDDVRIAVAGVFDYWGQGTLITVSS
213-C4K
(SEQ ID NO: 35)
QVQLVQSGGGVAQPGRSLRLSCAASGFTFSSFAIHWVRQAPGKGL
EWVAVISYDGGNKYYTDSVKGRFTISRDNSKNTLYLQMNSLKPED
TAVYYCARGYGGNYRGGDYWGQGTLVTVSS
213-C8K
(SEQ ID NO: 36)
QLQLQESGPGLVKPSETLSLTCTVSGGSISSYYWSWIRQPPGKGL
EWIAYMYYSGSTNFNPSLKSRVTISVDTSKNQFSLKLSSVTAADT
AVYYCARARPNLWFGAFDYWGQGTLVTVSS
213-D2L
(SEQ ID NO: 37)
QVQLVELGPGLVKPSETLSLTCTVSGGSISSYYWSWIRQPPGKGL
EWIAYMYYSGSTNFNPSLKSRVTISVDTSKNQFSLKLSSVTAADT
AVYYCARARPNLWFGAFDYWGQGTLVTVSS
213-D2k
(SEQ ID NO: 38)
QVQLVELGPGLVKPSETLSLTCTVSGGSISSYYWSWIRQPPGKGL
EWIAYMYYSGSTNFNPSLKSRVTISVDTSKNQFSLKLSSVTAADT
AVYYCARARPNLWFGAFDYWGQGTLVTVSS
213-D7L
(SEQ ID NO: 39)
QVQLVQSGGHVVQPGRSLRHSCAASGFTFSGYGMHWVRQAPGKGL
EWVAVIWYDGSNKFYADSVKDRFTISRDNSKNTLYLHMNSLRAED
TAVYYCARDSRELYYFDYWGQGTLVTVSS
213-D9L
(SEQ ID NO: 40)
EVHLVESGGGLVQPGGSLRLSCAASGFTFSSFWMTWVRQAPGKGL
EWVANIKEDGSEKYYVDSVKSRFTISRDNAKKPLYLQMNSLRAED
TAVYYCARDYTGSSFDFDNWGQGTLVTVSP
213-D11L
(SEQ ID NO: 41)
EVQLVQSGAEVKKPGASVKVSCKASGYTFNSDDINWVRQATGQGL
EWMGWMNPDSGNTGYAQKVQGRVTMTRNTSISTAYMELSSLRSED
TAVYYCAGTVSGYDFHSSYYYIDVWGKGTTVTVSS
213-E2L
(SEQ ID NO: 42)
QMQLVQSGGGVVQPGRSLRLSCAASGFTFSTYAFHWVRQAPGTGL
EWVAVMSYDGSNKYYADSVKGRFTISRDNSKNTLYLQMNSLRAED
TAVYYCARDSLYYYDNSAYLPHFDYWGQGTLVTVSS
213-E4k
(SEQ ID NO: 43)
QMQLVQSGAEVKKPGSSVKVSCKAAGGTFSSYAISWVRQAPGQGL
EWMGGIIPIFGTANYAQKFQGRVTITADKSTSTAYMELSSLRSED
TAVYYCAKGLGYYDSSGYTNWFDTWGQGTLVTVSS
213-E6k
(SEQ ID NO: 44)
EVQLLSGAEVRKPGSSVKVSCKASGGSFSSYGINWVRQAPGQGLE
WMGGIIPIFHTANYAQKFQGRVTITADESTSTANMEVRSLRSEDT
AIYYCVWFRELDNETPDDPGDYGLDVWGQGTTVTVSS
213-E11L
(SEQ ID NO: 45)
EVQLVQSGGGVVRPGGSLRLSCAASGFIFDDYGMNWVRQAPGKGL
EWVSGINWNGGRTGYADSVKGRFTISRDNAKNSLYLQMNSLRAED
TALYYCARGYCGGDCYSLTLDDAFDIWGQGTMVTVSS
213-F1k
(SEQ ID NO: 46)
QVQLLQPGGGVVQPGRSLRLSCAASGFTFSTYAFHWVRQAPGTGL
EWVAVMSYDGSNKYYADSVKGRFTISRDNSKNTLYLQMNSLRAED
TAVYYCARDSLYYYDNSAYLPHFDYWGQGTLVTVSS
213-GIL
(SEQ ID NO: 47)
EVHLVESGGGVVQPGRSLRLSCAASGFTFTNYGMHWVRQAPGKGL
EWVAVISFDGSNKYYADSVKGRFTISRDNSENTLYLQMNSLRAED
TAVYYCAKERGWPENSFDSWGQGTLVTVSS
213-G3k
(SEQ ID NO: 48)
EVQLLESGGGLVQPGGSLRLSCAASGFTFSVYDMHWVRQGTGKGL
EWVSAIDTAGDTFYPDSVKGRFTISRENAKSSLYLQINSLRAGDT
AVFFCARGRRGYYDRRGFYNWAGFDVWGHGTMVTVSS
213-G3L
(SEQ ID NO: 49)
EVQLLESGGGLVQPGGSLRLSCAASGFTFSVYDMHWVRQGTGKGL
EWVSAIDTAGDTFYPDSVKGRFTISRENAKSSLYLQINSLRAGDT
AVFFCARGRRGYYDRRGFYNWAGFDVWGHGTMVTVSS
213-G6k
(SEQ ID NO: 50)
QVQLQQSGPGLVKPSETLALTCTVSGGSTSSYYWSWIRQPPGKGL
EWIGSIYYSGSTDYNPSLKSRVTISLDTSKTQFSLKLSSVTAADT
AVYYCARVDTSGWYTGDVFDYWGQGTLVTVSS
213-G6L
(SEQ ID NO: 51)
QVQLQQSGPGLVKPSETLALTCTVSGGSTSSYYWSWIRQPPGKGL
EWIGSIYYSGSTDYNPSLKSRVTISLDTSKTQFSLKLSSVTAADT
AVYYCARVDTSGWYTGDVFDYWGQGTLVTVSS
213-G7L
(SEQ ID NO: 52)
EVQLVQSGAEVKKPGASVKVSCKVSGYTLIELSMHWVRQAPGKGL
EWMGGFDPEDAETIYAQEFQGRVTMTEDTSTDTAYMELSSLRSED
TGVYYCATSSAVIKGGWLDPWGQGTLVIVSS
213-G9k
(SEQ ID NO: 53)
EVQLVQSGAEVKKPGSSVKVSCKASGGTFSGYAISWVRQAPGQGL
EWMGGIIPFFGSAIYAQKFQGGVTITADESTSTASMELSSLRSED
TAVYYCARVGGIVATIPGAHYGMDVWGQGTTVTVSS
213-Hlk
(SEQ ID NO: 54)
QVQLLQPGAEVKKPGSSVKVSCKASGGTFRTYAISWVRQAPGQGL
EWMGGIIPIFGTANYAQKFQGRVTITADKSTSTAYMELTSLRSED
TAVYYCARLSATVLTPSYWGQGTLVTVSS
213-HIL
(SEQ ID NO: 55)
QVQLLQPGAEVKKPGSSVKVSCKASGGTFRTYAISWVRQAPGQGL
EWMGGIIPIFGTANYAQKFQGRVTITADKSTSTAYMELTSLRSED
TAVYYCARLSATVLTPSYWGQGTLVTVSS
213-H2k
(SEQ ID NO: 56)
QVQLQESGPGLVKPSQTLSLTCTVSGGSISSGSYYWSWIRQHPGK
GLEWIGYIYYSGGTYYNPSLKSRVTISLDTSKNQFSLKLSSVAAA
DTAVYYCAREGLRSEAGRGLLEYYGMDVWGQGTTVTVSS
213-H3L
(SEQ ID NO: 57)
QVQLVQSGPTLVKPTQTLTLTCTFSGFSLTTSGVNMGWIRQPPGK
ALEWLALIFWDDDKRYSASLKSRLAITKDTSKNQVVLTMTNMDPV
DTATYFCAHSLTMTTPAFDIWGPGTMVTVSS
Light chain sequence corresponding to the
tested sequences are described below:
Light Chains
001-A7k
(SEQ ID NO: 58)
DIQMTQSPSSLSASVGDRVTITCRASQSLSSYLNWYQQKPGKAPK
LLIYAVFSLQSGVPSRFSGSGSGTDFTLTISSLQPEDFATYYCQQ
SYSTPLTFGGGTKVETK
001-A8L
(SEQ ID NO: 59)
SYELTQPPSMSVSPGQTASITCSGDKLGDRYACWYQQKPGQSPVL
VIYQDSKRPSGIPERFSGSNSGNTATLTISGTQAMDEADYYCQAW
DSSTDVVFGGGTKLTVL
001-A10k
(SEQ ID NO: 60)
DIVMTQSPLSLPVSPGEPASISCRSSQSLLQSNGYNYLDWYLQKP
GQSPQLLIFLGSNRASGVPDRFSGSGSGTDFTLKISRVEAEDVGV
YYCMQGLQAPMYTFGQGTKLEIK
001-A11L
(SEQ ID NO: 61)
DIVMTQSPDSLAVSLGERATINCKSSQSVLYSSNNKNYLAWYQQK
PGQPPKLLIYWTSTGESGVPDRFSGSGSGTDFTLTISSLQAEDVA
VYYCQQYYSTPVTFGQGTRLEIK
001-A12L
(SEQ ID NO: 62)
SYELTQPPSVSVSPGQTASITCSGDKLGDKYACWYQQKPGQSPVV
VIYEDSKRPSGIPERFSGSNSGNTATLTISGTQAMDEADYYCQAW
DSSTAVFGGGTKLTVL
996-A7k
(SEQ ID NO: 63)
SYELTQPPSVSVAPGQTARITCGGNNIGGKSVHWYQQKPGQAPVL
VVYDDSDRPSGIPERFSGSSSGNTATLTISRVEAGDEADYYCQVS
DTSRDHRGVFGGGTKLTVL
996-A11k
(SEQ ID NO: 64)
DIQMTQSPSSLSASVGDRVIITCRASQGISNYLAWFQQKPGKAPK
SLIYAASSLQSGVPSKFSGSGSGTDFTLTISSLQPEDFGTYYCQQ
YNSYPLTFGGGTKVEIK
996-B1k
(SEQ ID NO: 65)
EIVLTQSPGTLSLSPGERATLSCRASQSVSSTYLAWYQQKPGQAP
RLLIYGASSRATGIPDRFSGSGSGTDFTLTISRLDPEDFAVYYCQ
QYGSSPPLTFGGGTKVEIK
996-C5L
(SEQ ID NO: 66)
QSALTQPASVSGSPGQSITISCTGTSSDIGGYDYVSWYQHHPGKA
PKLMIYDVSNRPSGVSNRFSGSKSGNTASLTISGLQAEDEADYYC
NSYTNISTLGVFGGGTKLTVL
996-C6k
(SEQ ID NO: 67)
DIVMTQSPLSLPVTPGEPASISCRSSQSLLHSNGYNYLDWYLQKP
GQSPQLLIYLGSNRASGVPDRFSGSGSGTDFTLKISRVEAEDVGV
YYCMQALQTPGTFGQGTKVEIK
996-C7k
(SEQ ID NO: 68)
DIQMTQSPSSLSASVGDRVIITCRASQSISSYLNWYQQKPGKAPK
LLIFAASSLQGGVPSRFSGSGSGTDFTLTISSLQPEDFAIYYCQQ
SYVNFPITFGQGTRLEIK
996-C9L
(SEQ ID NO: 69)
QSALTQPRSVSGSPGQSVTISCTGTSSDVGGYNYVSWYQQHPGKA
PKFMIYDVSKRPSGVPDRFSGSKSGNTASLTISGLQAEDEADYYC
CSYAGSLWVFGTGTKVTVL
206-A1L
(SEQ ID NO: 70)
EIVLTQSPVTLSLSPGERATLSCRASQSVSSSYLAWYQQKPGQAP
RLLIYGASSRTTGIPDRFSGSGSGTDFTLAISRLEPEDFAVYYCQ
QYGSSPQTFGQGTKVEIK
206-A8L
(SEQ ID NO: 71)
VIWMTQSPSFLSASVGDRVTITCRASQGISSYLAWYQQKPGKAPK
LLIYAASTLQSGVPSRFSGSGSGTEFTLTISSLQPEDFATYYCQQ
LNSYPLFGPGTKVDIK
206-A9k
(SEQ ID NO: 72)
AIRMTQSPSTLSVSPGERATLSCRASQSVSSYLAWYQHKPGQAPR
LLVYGASTRATGIPARFSGSGSGTEFTLTISSLQSEDFAVYYCQQ
YNNWPPPFTFGPGTKVDIK
206-B10L
(SEQ ID NO: 73)
DIVLTQSPVTLSLSPGERATLSCRASQSASSYLAWYQQKPGQAPR
LLIYDASKRATGIPARFSGSGSGTDFSLTISSLEPEDFAVYYCQQ
RSNRPPTWTFGQGTKVEIK
206-C8K
(SEQ ID NO: 74)
QSALTQPASVSGSPGQSITISCTGTSSDVGGYNYVSWYQQHPGKA
PKLMIYEVRNRPSGVSHRFSGSKSGNTASLTISGLQAEDEADYYC
SSKTSSSTLYVFGTGTKVTVL
206-C10k
(SEQ ID NO: 75)
QAVLTQPTSLSASPGASARFTCTLRSGINVGTYRIYWYQQKPGSL
PRYLLRYKSDSDYQRGSGVPSRFSGSKDASTNAGLLLISGLQSED
EADYYCAIWYSSTLVFGGGTKLTVL
206-D5L
(SEQ ID NO: 76)
QSALTQPRSVSGSPGQSVTISCTGTSSDVGGYNYVSWYQQYPGKA
PKLMIYDVSKRPSGVPDRFSGSKSGNTASLTISGLQGEDEADYYC
CSYAGSYTYVFGTGTKVTVL
206-Elk
(SEQ ID NO: 77)
QSALTQPPSVSVSPGQTARITCSGDALPKKYAYWYQQKSGQAPVL
VIYEDSKRPSGIPERFSGSSSGTMATLTISGAQVEDEADYYCYST
DTSGNRWVFGGGTKLIVL
208-A7k
(SEQ ID NO: 78)
AIRMTQSPSTLSVSPGERATLSCRASQSVSSNLAWYQQKPGQAPR
LLIYGASTRATGIPVRFSGSGSGTESTLTISSLQSEDFAVYYCQQ
YNKWPRTFGQGTKVEIK
208-A11k
(SEQ ID NO: 79)
EIVMMQSPVSLPVTLGQPASISCRSSQSLVHSDGNTYLNWFQQRP
GQSPRRLIYKVSNRDSGVPDRFSGSGSGTDFTLKISRVEAEDVGV
YYCMQGTHWPRTFGGGTKVEIK
208-B2L
(SEQ ID NO: 80)
QLVLTQSSSASASLGASVKLTCTLTSGHSSYAIAWHQQQPEKGPR
YLMKLNSDGSHSKGDGIPDRFSGSSSGADRYLTISSLQSEDEADY
YCQTWGTGVQVFGGGTKLTVL
208-C12L
(SEQ ID NO: 81)
SYELTQPPSVSVSPGQTARITCSGDALPKKYAYWYQQKSGQAPVL
VIYEDRKRPSGIPERFSGSSSGTMATLTISGAQVEDEADYYCFST
DSSDNQRVFGGGTKLTVL
208-D3k
(SEQ ID NO: 82)
DVVMTQSPLTLSLSPGERATLSCRASQSVSSNYLAWYQQKPGQAP
RLLIYGASSRATGIPDRFSGSGSGTDFTLTISRLEPEDFAVXYCQ
QYGSSYTFGXXTKLEIK
208-D11L
(SEQ ID NO: 83)
QSALTQSPSASASLGVSVKLTCTLTSGHSSYAIAWHQQQPEKGPR
YLMKLNSDGSHSKGDGIPDRFSGSSSGADRYLTISSLQSEDEADY
YCQTWGTGVQVFGGGTKLTVL
208-F1k
(SEQ ID NO: 84)
EIVLTQSPVTLSVSPGERATLSCRASQSVGSNLAWYQQKPGQAPR
LLIYGASTRATGIPARFSGSGSGTEFTLIISSLQSEDFAVYYCQQ
YNNWPPLLTFGGGTKVEIK
213-A1L
(SEQ ID NO: 85)
QSALTQPASVSGSPGQSITISCTGTSSDVGSYNLVSWYQQHPGKA
PKLMIYEAEDEADYYCCSYAGPWVFGGGTKLTVL
213-A3L
(SEQ ID NO: 86)
QAGLTQPASVSGSPGQSITISCTGTSSDVGSYNLVSWYQQHPGKA
PKLMIYDVSKRPSGVSNRFSGSKSGNTASLTISGLQAEDEADYYC
CSYAGRSTFPYVFGTGTKLTVL
213-A5L
(SEQ ID NO: 87)
RPVLTQPPSTSGTPGQRVTISCSGSSSNIGSNTVNWYQHLPGTAP
KLLIYSNHQRPSGVPDRFSGSKSGTSASLAISGLQSEDEADYYCA
TWDDSLNGWLFGGGTKLTVL
213-A9L
(SEQ ID NO: 88)
QSVLTQPPSVSVSPGQTARITCSGDALPKQYAYWYQQKPGQAPVM
LIYKDSERPSGIPERFSGSSSGTTVTLTISGVQAEDEADYYCQSA
DSSGTVVFGGGTKLTVL
213-A11L
(SEQ ID NO: 89)
QAVLTQPHSVSESPGKTVTISCTGSSGSIASNYVQWYQQRPGSAP
TTMIYEDNQRPSGVPDRFSGSIDSSSNSASLTVSGLKTEDEADYY
CQSYDSSNLWVFGGGTKLTVL
213-B6k
(SEQ ID NO: 90)
DVVMTQSPLTLSLSPGERAILSCRASQSVYSSYLAWYQQKPGQAP
RLLIYGASSRATGIPDRFSGSGSGTDFTLTISRLEPEDFAVYYCQ
QYGSSPPVYAFGQGTKLEIK
213-B12L
(SEQ ID NO: 91)
RPVLTQPPSLSVSPGQTASITCSGDKLGDKYSSWYQQKPGQSPVL
VIYQDTKRPSGIPERFSGSNSGNTATLTISGTQAMDEADYYCQAW
DSSNVVFGGGTKLTVL
213-C4K
(SEQ ID NO: 92)
AIRMTQSPSSLSASVGDRVTITCRASQSISSYLNWYQQKPGKAPK
FLIYAASSLQSGVPSRFSGSGSGTDFTLTISSLQPEDFATYYCQS
SYSTRWTFGQGAKVEIK
213-C8K
(SEQ ID NO: 93)
AIRMTQSPSSLSAFVGDRVTITCRASQNLSNYLNWYQHKPGKAPN
LLIYAASSLQSGVPSKFSGSGSVTDFTLTISSLQPEDVGTYYCQQ
TYSVPLTLGPGTKVHII
213-D2L
(SEQ ID NO: 94)
QAGLTQPPSVSVSPGQTARITCSGDALPKQYAYWYQQKPGQAPVM
LIYKDSERPSGIPERFSGSSSGTTVTLTISGVQAEDEADYYCQSA
DSSGTVVFGGGTKLTVL
213-D2k
(SEQ ID NO: 95)
DIVMTQTPLSLSASVGDRVTITCRASQSISNYLSWYQQKPGKAPK
LLIYAASSLQSGVPSRFSGSGSGTDFTLTISSLQPEDFAIYYCQQ
SYSTLTWTFGQGTKVEIK
213-D7L
(SEQ ID NO: 96)
RPVLTQPPSVSVSPGQTASITCSGDKLGDKYACWYQQKPGQSPVV
VIYQDTKRPSGIPERFSGSNSGNTATLTISGTQAMDEADYYCQAW
DSFTSVVFGGGTKLTVL
213-D9L
(SEQ ID NO: 97)
QAVLTQPRSVSGSPGQSVTISCTGTSSDVGGYNSVSWYQQHPGKA
PKLMIYDVTKRPSGVPDRFSGSKSGNTASLTISGLQAEDEADYYC
CSYAGRYLYVFGTGTKVTVL
213-D11L
(SEQ ID NO: 98)
QSALTQPPSTSGTPGQRVTISCSGSSSNIGSNTVNWYQHLPGTAP
KLLIYSNHQRPSGVPDRFSGSKSGTSASLAISGLQSEDEADYYCA
TWDDSLNGWLFGGGTKLTVL
213-E2L
(SEQ ID NO: 99)
SYELTQPPSVSAAPGQRVTISCSGSSSNIGNNYVSWYQQFPGTAP
KLLIYDNNERPSGIPDRFSGSKSGTSATLGITGLQAGDEADYYCG
TWDSSLSAVYVFGTGTKVSVL
213-E4k
(SEQ ID NO: 100)
EIVLTQSPLTLSLSPGERATLSCRASQSIRSTYFAWYQQKPGQAP
RLLIYGASSRATGIPDRFSGSGSGTDFTLTISRLEPEDFAVYYCQ
QYGSSPLTFGGGTKVEIK
213-E6k
(SEQ ID NO: 101)
EIVLTQSPVTLSLSPGERATLSCRASQSVSSFLAWYQQKPGQPPK
LLIYDASNRATGIPARFSGRGSGTDFTLTISSLEPEDFAVYYCQR
RSNWPPYTFGQGTKLEIK
213-E11L
(SEQ ID NO: 102)
RPVLTQPPSVSGAPGQRVTISCTGSSSDDVHWYQQLPGTAPKLLI
YGNTNRPSGVPDRFSGSKSGTSASLAITGLQAEDEADYYCQSYDS
SLSGSVVFGGGTKLTVL
213-F1k
(SEQ ID NO: 103)
EIVLTQSPVTLSVSPGESATLSCRASQSVSSNLAWYQQKLGQAPR
LLIYGASIRATGFPARFSGSGSGTEFTLTISSLQSEDFAVYYCQQ
YDNWPPWTFGQGTKVEIK
213-GIL
(SEQ ID NO: 104)
QPVLTQPPSVSVSPGQTARITCSGDKLGDKYACWYQQKPGQSPVL
VIYQDSQRPSGIPERFSGSSSGNTATLTISGTQAMDEADYYCQAW
DSSTVVFGGGTKLTVL
213-G3k
(SEQ ID NO: 105)
DIVMTQSPLTLSVSPGERATLSCRASQSVSRSVAWYQQKPGQAPR
LLMYGASTRATGTPARFSGSGSGTEFTLTISSLQSEDFAVYHCQQ
YDNRPPWTFGRGTKVEIK
213-G3L
(SEQ ID NO: 106)
QAGLTQPPSVSVAPGQTARITCGGNNIGSKSVHWYQQKPGQAPVL
VVYDDSDRPSGIPERFSGSNSGNTATLTISRVEAGDEADYYCQVC
DSTSDHVFGTGTKVTVL
213-G6k
(SEQ ID NO: 107)
AIQMTQPPSSLSASVGDRVTITCRASQSLSSYLNWYQQKPGKAPK
LLIYAVFSLQSGVPSRFSGSGSGTDFTLTISSLQPEDFATYYCQQ
SYSTPLTFGGGTKVEIK
213-G6L
(SEQ ID NO: 108)
QSALTQPASVSGSPGQSITISCTGTSSDVGGYNYVSWYQQHPGIA
PKLMIYDVNKRPSGVPDRFSGSKSGNTASLTISGLQAEDEADYYC
SSYTSSSTVVFGGGTKLTVL
213-G7L
(SEQ ID NO: 109)
QSALTQPASVSGSPGQSITISCTGTSSDVGGYNYVSWYQQHPGKA
PKLMIYDVTNRPSGISDRFSGSKSGNTASLTISGLQAEDEADYYC
SSYTSTSTLVFGGGTKLTVL
213-G9k
(SEQ ID NO: 110)
DIQMTQPPSSLSASVGDRVTITCQASQDISTYLNWYQQKSGKAPK
LLIYDASNLETGVPSRFSGSGSGTDFTFTISSLQPEDIATYYCQQ
YDDLPTFGGGTKVEIK
213-H1k
(SEQ ID NO: 111)
DVVMTQSPLSLPVSPGEPASISCRSSQSLLQSNGYNYLDWYLQKP
GQSPQLLIFLGSNRASGVPDRFSGSGSGTDFTLKISRVEAEDVGV
YYCMQGLQAPMYTFGQGTKLEIK
213-HIL
(SEQ ID NO: 112)
QAGLTQPPSASGTPGQTVTISCSGSSSNIGSNTVNWYQQLPGTAP
KLVIFTNNQRPSGVPDRFSGSTSGTSASLAISGLQPEDEADYYCA
TWDDSLNGVVFGGGTTLTVL
213-H2k
(SEQ ID NO: 113)
VIWMTQSPSTLSVSPGERATLSCRASQSVSSNLAWYQQKPGQAPR
LLIYGASTRATGIPARFSGSGSGTEFTLTISSLQSEDFAVYYCQQ
YNNWPPWTFGQGTKVEIK
213-H3L
(SEQ ID NO: 114)
QSVLTQPASVSGSPGQSITIACTGSSSDVGGHNYVSWYQQHPGKA
PKLMIYEVINRPSGVSNRFSGSKSGNTASLTISGLQAEDEADYYC
SSYRSSSPVVFGGGTKLTVL

### Example 2. Neutralization of SARS-COV2 Pseudovirions

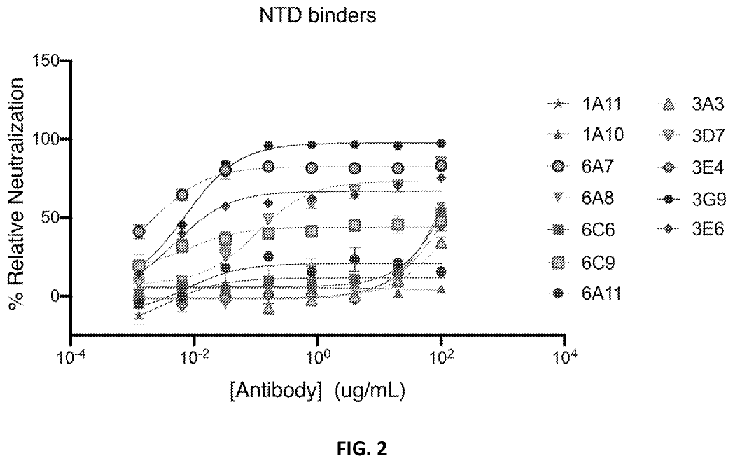

Pre-titrated amounts of rVSV-SARS-COV-2 was incubated with serially diluted purified monoclonal antibody at 37 C for 1 hr before addition to confluent Vero monolayers in 96-well plates. Infection proceeded for 12-16 hrs at 37° C. in 5% CO₂ before cells were fixed in 4% paraformaldehyde and stained with 10 ug/mL Hoechst. Cells were imaged using a CellInsight CX5 imager and infection was quantitated by automated enumeration of total cells and those expressing GFP. Infection was normalized to the average number of cells infected with rVSV-SARS-COV-2 incubated with normal human sera. Data are presented as the relative neutralization for each concentration of antibody. Neutralization IC50s (displayed as ug/mL) were calculated using “One-Site Fit LogIC50” regression in GraphPad Prism 9.0. The data is summarized in FIG. 2 and Table 1.

TABLE 1

| | | IC50 | Maximum neutralization |
|---|---|---|---|
| | | (ug/mL) | at 100 ug/mL [Ab] |
| NTD | 6A11 | NN | NN |
| binders | 6C6 | 296.36 | 53.18 |
| | 1A10 | NN | NN |
| | 6A8 | 214.76 | 57.00 |
| | 3E6 | 0.005* | 75.54 |
| | 6A7 | 0.0024* | 83.22 |
| | 6C9 | 0.008* | 47.70 |
| | 3A3 | >100 | 34.25 |
| | 3D7 | 0.105* | 85.55 |
| | 3E4 | 106.90 | 45.01 |
| | 3G9 | <0.007 | 97.20 |
| | 1A11 | NN | NN |

*a variable fraction of virus is left un-neutralized for these antibodies

### Example 3. Binding and Kinetic Analysis of Anti-SARS-COV-2 Spike Antibodies

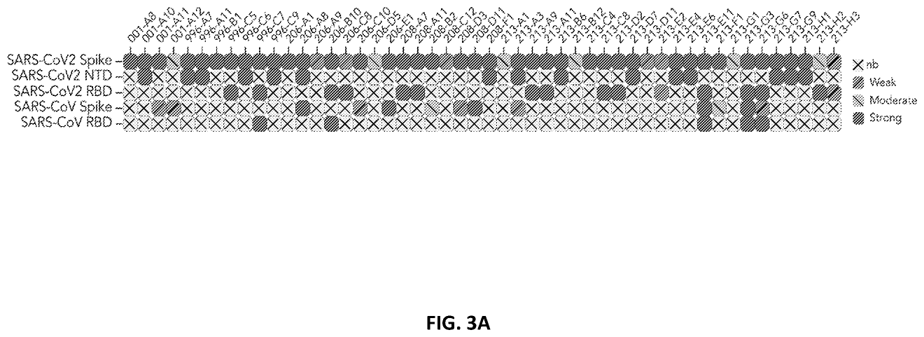

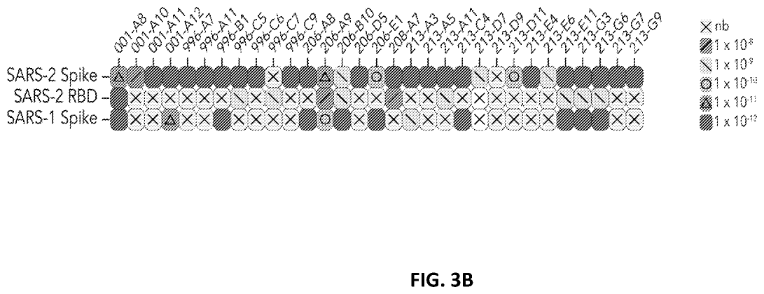

Certain antibodies detailed herein were tested against different SARS proteins to determine their binding and kinetic. The reactivity of each antibody for the indicated protein was determined. This data is summarized in FIG. 3A. The binding affinity for the indicated antibody-protein pair was also determined using biolayer interferometry. This data is summarized in FIG. 3B.

### Example 4. Neutralization of SARS-COV-2 Pseudovirions by Select Antibodies

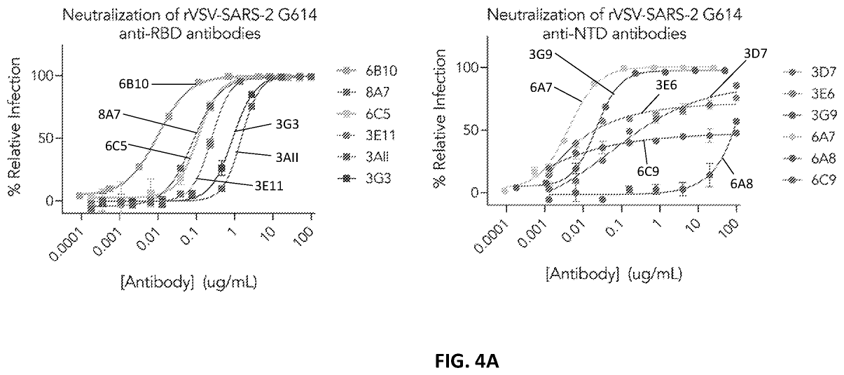

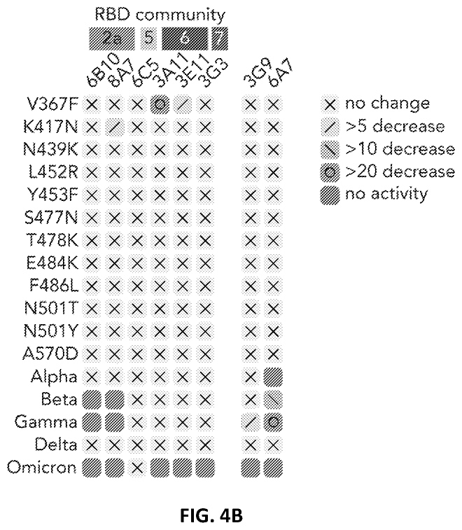

Certain antibodies detailed herein were tested to determine if they could neutralize SARS-COV-2 pseudovirions. Neutralization of rVSV-based SARS-COV2 (G614 variant) pseudoparticles by receptor-binding domain (RBD) or N-terminal domain (NTD) specific antibodies was determined. This data is summarized in FIG. 4A. Fold-change differences in neutralization potency for select antibodies was also determined. This data is summarized in FIG. 4B. Pseudovirions were produced with either the single point mutation indicated or with the full constellation of mutations represented in major variants of concern.

### Example 5. Structural Characteristics of Anti-SARS-COV-2 Antibodies

Certain antibodies detailed herein were structurally characterized as they were bound to spike proteins of SARS-COV-2. Negative stain electron microscopy (nsEM) reconstructions of the indicated RBD-specific antibody bound to the trimeric Spike protein were determined. The Spike is shown as a surface representation with each monomer colored white, gray, or black. The bound antibody is colored accordingly. This data is summarized in FIGS. 5A-5B. High-resolution crystal structures of the Fab of 6B10 or 6C5, as indicated, bound to the RBD of SARS-COV2 were determined. This data is summarized in FIG. 5C.

### Example 6. Protection of K18-hACE2 tg Mice Against SARS-COV-2 Infection

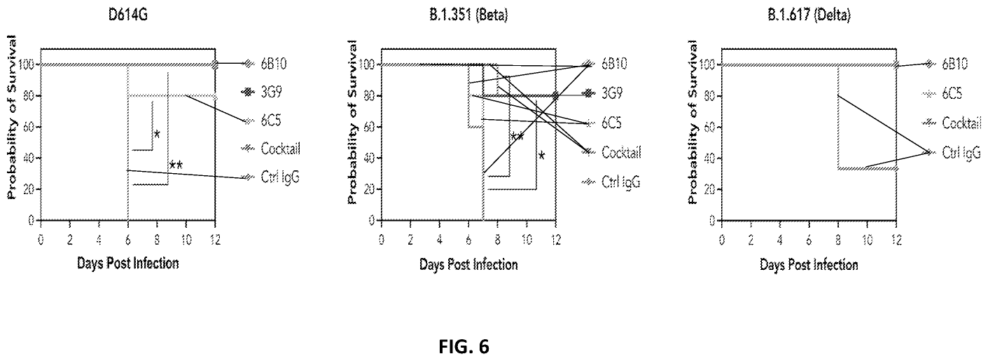

Certain antibodies detailed herein were tested to determine whether they could protect K18-hACE2 tg mice against SARs-COV-2 infection. Individual antibodies at a concentration of 10 mg/kg were delivered intraperitoneally one day prior to infection with 104 pfu of the indicated virus. A cocktail of three (G614 or B.1.351) or two (B.1.617) was also tested. A non-SARS-COV2 specific antibody was used as a control. The data is summarized in FIG. 6.

What is claimed:

1. An antibody composition comprising a heavy chain having at least 80%, or at least 85%, or at least 90%, or at least 95% sequence identity with any one of SEQ ID NOs: 1-57.

2. The antibody composition of claim 1, wherein the heavy chain comprises any one of SEQ ID NOs: 1-57.

3. An antibody composition comprising a light chain having at least 80%, or at least 85%, or at least 90%, or at least 95% sequence identity with any one of SEQ ID NOs: 58-114.

4. The antibody composition of claim 3, wherein the light chain comprises any one of SEQ ID NOs: 58-114.

5. An antibody composition comprising:
(a) a heavy chain having at least 80%, or at least 85%, or at least 90%, or at least 95% sequence identity with any one of SEQ ID NOs: 1-57; and
(b) a light chain having at least 80%, or at least 85%, or at least 90%, or at least 95% sequence identity with any one of SEQ ID NOs: 58-114.

6. The antibody composition of claim 5, wherein:
(a) the heavy chain comprises any one of SEQ ID NOs: 1-57; and
(b) the light chain comprises any one of SEQ ID NOs: 58-114.

7. The antibody composition of claim 1, wherein the antibody composition is specific for a spike protein of SARS-COV-2

8. The antibody composition of claim 1, wherein the antibody composition is specific for a receptor binding domain of SARS-COV-2.

9. The antibody composition of claim 1, wherein the antibody composition comprises an antibody or a fragment thereof that is specific for an N-terminal domain of SARS-COV-2.

10. A method of treating an individual infected with SARS-COV-2, the method comprising administering to the individual an effective amount of an antibody composition according to any one of claims 1-9.

11. A method of preventing SARS-COV-2 infection in an individual, the method comprising administering to the individual an effective amount of an antibody composition according to any one of claims 1-9.

12. The method of claim 10 or 11, wherein the antibody composition is administered together with an adjuvant.

13. The method of claim 10 or 11, wherein the antibody composition binds to a receptor binding domain of SARS-COV-2.

14. The method of claim 10 or 11, wherein the antibody composition binds to a spike protein of SARS-COV-2.

15. The method of claim 10 or 11, wherein administering of the antibody composition results in neutralization of SARS-COV-2 pseudovirions.

16. A method of identifying SARS-COV-2 in an individual, the method comprising:
(a) isolating a biological sample from the individual;
(b) incubating the biological sample with an antibody composition according to any one of claims 1-9; and
(c) detecting a biological interaction between the biological sample and the antibody composition.

17. A kit for identifying SARS-COV-2 in a biological sample, the kit comprising:
(a) an antibody composition according to any one of claims 1-9; and
(b) instructions of use.

18. An enzyme-linked immunosorbent assay (ELISA) test kit comprising:
(a) an antibody composition according to any one of claims 1-9; and
(b) instructions of use.

19. Use of an antibody composition according to any one of claims 1-9 for treating SARS-COV-2.

20. Use of an antibody composition according to any one of claims 1-9 to formulate a medicament for treating SARS-COV-2.
